(19) United States
(12) Patent Application Publication
(10) Pub. No.: US 2008/0242727 A1
(43) Pub. Date: Oct. 2, 2008

(54) DIETARY COMPOSITIONS CONTAINING ALPHA AMINO N-BUTYRATE AND METHODS OF ENHANCING LEAN BODY MASS

(76) Inventors: Tim Romero, Sarasota, FL (US); Peter Miller, Broomfield, CO (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/056,662

(22) Filed: Mar. 27, 2008

Related U.S. Application Data
(60) Provisional application No. 60/908,204, filed on Mar. 27, 2007.

(51) Int. Cl. A61K 31/164; A61P 21/00

(52) U.S. Cl. 514/561

(57) ABSTRACT

A composition that may include branched chain amino acids, alpha amino n-Butyrate and/or alpha amino-n-valerate works synergistically to enhance lean body mass and prevent body mass breakdown. The composition may also be coupled with other agents to a) provide an increased level of amino acids and/or protein in the body's total pool or b) increase markers for protein translation or decrease markers of protein turnover. The composition may be administered in a variety of ways including capsules, tablets, powdered beverages, bars, gels or drinks. Increasing lean body mass is import to athletes looking to enhance performance, in the event of certain muscle wasting diseases, and to the general population that loses muscle mass as it ages.

### CROSS-REFERENCE TO RELATED APPLICATIONS

The present application claims priority from U.S. Provisional Application No. 60/908,204, filed Mar. 27, 2007, the disclosure of which is incorporated by reference herein.

### FIELD OF THE INVENTION

Disclosed herein is an embodiment(s) of a dietary supplement comprising alpha amino n-butyrate or alpha amino n-valerate, or both in combination, with or without a branched chain amino acid(s). Also disclosed are methods of enhancing protein synthesis and decreasing protein catabolism, both in humans and animals, for the purpose of enhancing lean body mass and exercise performance, combating muscle wasting associated with dieting and/or disease. The supplement may also include additional components that provide a greater level of amino acids and/or protein in the body's total pool, or increase markers for protein translation, or decrease markers of protein turnover.

### BACKGROUND OF THE INVENTION

Branched chain amino acids, particularly the amino acid L-Leucine, are known for their beneficial properties. Leucine, for example, preserves muscle and protein synthesis and decreases protein breakdown during times of weight loss or other catabolic circumstances. In 2001, Anthony et al. reported that Leucine also controls protein turnover in muscle at the level of translation initiation.

On the other hand, in 1996 Nissen et al., and then in 2000 Gallagher et al., reported that a dose of as little as 3 g/day of the leucine metabolite, HMB (b-hydroxy b-methylbutyrate) is anti-catabolic, promotes lean muscle mass, and may speed recuperation. When combined with the other branched-chain amino acids (BCAAs) valine and isoleucine, a dose of 10 g+/day of Leucine have also been shown to be anabolic. Furthermore, a dose of 5.5 g+/day may speed recuperation.

In 2006, Layman evaluated the effects of exercise on branched chain amino acid infusion as it relates to insulin and muscle protein synthesis. Layman, for example reported that:
During exercise, muscle protein synthesis decreases together with a net increase in protein degradation and stimulation of BCAA oxidation. The decrease in protein synthesis is associated with inhibition of translation initiation factors 4E and 4G and ribosomal protein S6 under regulatory controls of intracellular insulin signaling and leucine concentrations. BCAA oxidation increases through activation of the branched-chain alpha-keto acid dehydrogenase (BCKDH). BCKDH activity increases with exercise, reducing plasma and intracellular leucine concentrations. After exercise, recovery of muscle protein synthesis requires dietary protein or BCAA to increase tissue levels of leucine in order to release the inhibition of the initiation factor 4 complex through activation of the protein kinase mammalian target of rapamycin (mTOR). Leucine's effect on mTOR is synergistic with insulin via the phosphoinositol 3-kinase signaling pathway. Together, insulin and leucine allow skeletal muscle to coordinate protein synthesis with physiological state and dietary intake.
   Accordingly, with exercise, the demand for leucine in particular increases as demand for protein synthesis increases.

In 2003, Layman suggested that in addition to functioning as a substrate for protein synthesis, leucine is also a precursor for alanine, and a modulator of muscle protein synthesis via the insulin-signaling pathway. More specifically, the greater the intracellular BCAA concentrations, the more circulating alanine that can be taken up by the liver to support hepatic gluconeogenesis. Thus, a high protein diet, rather than a high carbohydrate diet, will reduce the role of insulin in managing acute changes in blood glucose and maximize the liver's role in regulating blood glucose. Leucine stimulates protein synthesis during catabolic states. As such, dietary levels of leucine influence maintenance of muscle mass during weight loss or other catabolic circumstances such as a disease state involving muscle wasting.

In 2005, Garlick evaluated Leucine's role in enhancing protein synthesis. This evaluation indicated that the physiological role of leucine was to work synergistically alongside insulin to activate the switch that stimulates muscle protein synthesis when amino acids and energy from food become available. Garlick also focused on the mode of regulation requiring both the necessary leucine and insulin to be present in order to activate the mechanism.

In 1998 Berning and Steen reported that for muscle mass to be enhanced, a person must be in a state of positive nitrogen balance. Several commentators have also suggested that the RDA for protein is not sufficient to maintain or increase nitrogen balance in athletes and bodybuilders. In addition, Friedman and Lemon in 1989; Tarnopolsky et al. in 1988; Tarnopolsky et al. in 1992; Lemon in 2000; Motil et al. in 1981; Tome and Bos in 2000; Meredith et al. in 1989; and Tipton et al. in 1999), which have compared nitrogen balance after different doses of protein in volunteers, support a conclusion that higher protein levels equals a positive nitrogen balance

Two of the formulas used to calculate metabolic rates include the formula reported by Cunningham in 1980: BMR (basic metabolic rate in cal/day)=500+22LBM; and the formula reported by Owen et al., in 1987: RMR (resting metabolic rate)=290+22.3 FFMD (fat-free mass) kg.

According to these formulas, the more lean body mass a person has, the more energy that person expends. If caloric intake remains the same as lean body mass increases, more energy is expended and body weight will decrease. This primarily relates not only to the amount of lean body mass a subject has, but rather to the implication of adipose tissue as it relates to sport and general well being.

In 1992, Nair et al. infused Leucine into the forearm vein of six human volunteers. As a result, the volunteers exhibited decreased plasma concentrations of several amino acids. The volunteers also decreased whole body valine flux and valine oxidation.

In 1997, Hoffer et al., reported that a second infusion in seven human volunteers confirmed that leucine decreased whole body proteolysis. Furthermore, in 1990, Hood and Terjung found that calculations based on steady-state rates of leucine oxidation at rest and during exercise indicate that the recommended dietary intake of leucine were inadequate, since it is lower than measured whole-body rates of leucine oxidation. This inadequacy is exacerbated in individuals who are physically active.

In 1999, Anthony et al. administered Leucine by oral gavage to rats after exercise stimulated muscle synthesis independent of increased plasma insulin. In 2000a, Anthony et al. reported that oral administration of leucine stimulated protein synthesis in skeletal muscle of food-deprived rats above saline-treated controls. However, valine and isoleucine were ineffective. Oral administration of leucine to food deprived rats, with or without carbohydrates, restored protein synthesis equal to that in fed rats.

In 2000b, Anthony et al. reported that Leucine stimulated protein synthesis by enhancing eukaryotic initiation factor (eIF)4F formation independently of increases in serum insulin. Further, in 2000 Shah et al. report that oral leucine restored glucocorticoid-induced decreases in protein synthesis and mRNA translation in rats.

In 1975, Buse and Reid., in an in vitro study found that leucine increased the specific activity of muscle proteins during incubation with [14C]lysine in hemidiaphragms from fed or fasted rats incubated, with or without insulin (evidence of increased protein synthesis), while valine and isoleucine (the other BCAAs) had no effect or inhibitory effects, respectively. These same authors also found evidence that leucine prohibited protein degradation. Leucine, but not valine or isoleucine, decreased the negative nitrogen balance that was characteristic of hearts perfused with buffer that contained glucose and normal plasma levels of other amino acids, except for the BCAA.

However, in 1979 Chua et al., indicated that in the presence of leucine concentrations that resulted in maximal inhibition of protein degradation, the rate of protein synthesis was only 50% of the rate of proteolysis. Further, in 1982 Poso found that out of 12 amino acids tested, leucine was the strongest inhibitor of deprivation-induced rat liver proteolysis, but only at concentrations much higher than physiological concentrations.

As indicated supra, branched chain amino acids have a physiological role in protein synthesis. However, many researchers also suggest that the role of leucine in protein synthesis may simply be counterbalanced by its elevated oxidation level. For example, in 1998 Forslund demonstrated that though a large bolus of leucine was consumed, 24 hour anabolism was not enhanced nor was nitrogen balance. Likewise in 1997 el-Khoury reported that exercise did not stimulate enhanced nitrogen retention as seen in.

Accordingly, neither leucine infusion nor exercise stimulates an increase in the translational machinery that would lead to a viable, elevated level of nitrogen retention. Hence, neither leucine, or branched chain amino acid infusion, or exercise is enough to induce appreciable gains in lean body mass. Accordingly, it would be advantageous to provide for a viable method of enhancing or even maintaining lean body mass, be it for bodybuilder's, muscle wasting syndrome's or dieters.

In 1988, Constantoulakis et al., found that alpha amino n-butyrate (ABA) may function to increase hemoglobin content. ABA, for example, stimulated the growth of all classes of erythroid progenitors in vivo or in culture. Therefore as it pertains to lean body mass enhancement and preservation of muscle, ABA works to increase the oxygen carrying capacity of the body, thus serving to all for greater protein synthesis to occur.

Alpha amino n-Butryate and alpha amino n-valerate are non-essential amino acids. Both serve as a transaminative product of 2-Oxobutyrate. Further, a study by Bigelis et al., in 1983 suggested that alpha amino n-butyrate may spare the demand for leucine as they are structurally similar and may compete for uptake.

### SUMMARY OF THE INVENTION

Disclosed herein is an embodiment(s) for a dietary composition including alpha amino n-butyrate and/or n-valerate, or a combination of both, with or without a branched chain amino acid(s). Also disclosed are methods of enhancing protein synthesis and decrease protein catabolism, both in humans and animals for the purpose of enhancing lean body mass and exercise performance, and combating muscle wasting associated with dieting and/or disease. When administered in physiologically acceptable amounts, the disclosed methods and dietary composition enhance exercise performance and muscle growth through the enhancement of protein synthesis and decrease in muscle protein breakdown.

### DETAILED DESCRIPTION OF THE PREFERRED EMBODIMENTS

Disclosed herein is a dietary supplement composition effective at increasing protein synthesis and decreasing the oxidative catabolism. The disclosed supplement may include alpha amino-n butyrate, alpha amino-n-valerate, or a combination of both. More particularly, the composition may include alpha amino n-butryate or alpha amino n-valerate, or a combination thereof, in combination with another branched chain amino acid. Examples of acceptable branched chain amino acids include Leucine, Isoleucine and Valine.

The disclosed supplemental may be prepared in a variety of forms including, for example, as a powder, a liquid, a tablet, a capsule, a pill, a candy, a confection food additive or a gel cap. Further, the composition may be form by blending or chemically bonding of the amino n-Butryate, alpha amino n-valerate, or the combination thereof. The composition including the branched chain amino acid(s) may likewise be formed by blending or chemical bonding.

The concentrations of alpha amino-n butyrate and alpha amino-n-valerate as used in the disclosed composition may each be in a range between 1 g-10,000 g. However, more preferred ranges for alpha amino-n butyrate and alpha amino-N-valerate are from 100 g-5000 g. Still more preferred ranges are from 500 g-3000 g.

In addition to be used in combination with another branched chain amino acid, the alpha amino n-butyrate and/or alpha amino n-valerate composition may be combined with other amino acids or anabolic agents in which a synergy may exist. Examples of these other amino acids or anabolic agents include creatine, glutamine, threonine, methionine, tyrosine, alanine and other compounds. Creatine and glutamine, for example have unique synergies with the composition. Specifically, since the nature of creatine is to promote an anabolic state and that of glutamine is to prevent a catabolic state, combining each of these individually or in combination with either alpha amino n-butryate or valerate provides synergisms due to the mechanistic pathway of preventing or sparing leucine pools.

The molar ratio of the branched chain amino acid(s) and alpha amino n-butyrate may be 1:1 or 2:1. Likewise, the molar ratio of the branched chain amino acid(s) and alpha amino n-valerate may be 1:1 or 2:1.

In addition to the supplement disclosed above, it will be appreciated that the supplement may also include or consist of: 1) a salt or other derivate of alpha amino n-butyrate or alpha-amino-n-valerate; 2) an ester or ether derivate of alpha amino n-butyrate or alpha-amino-N-valerate. Similarly, the branched chain amino acid may include a salt or other derivate, or an ester or ether derivate of the branched chain amino acid.

Example formulations for the disclosed dietary supplement are provided infra:

EXAMPLE 1

| | |
|---|---|
| Leucine alpha amino n-butyrate | 2,000 mg |
| Isoleucine alpha amino n-butyrate | 1,000 mg |
| Valine alpha amino n-butyrate | 1,000 mg |

EXAMPLE 2

| | |
|---|---|
| Leucine alpha amino n-butyrate | 5,000 mg |
| Creatine Monohydrate | 5,000 mg |
| Beta-Alanine | 1,000 mg |
| 4-hydroxyIsoleucine | 200 mg |

EXAMPLE 3

| | |
|---|---|
| alpha amino n-butyrate | 1,000 mg |

EXAMPLE 4

| | |
|---|---|
| alpha amino n-butyrate | 1,000 mg |
| beta-hydroxy beta-methyl butyrate | 3,000 mg |
| arginine alpha-ketogluturate | 3,000 mg |
| arginine keto-isocaproate | 500 mg |

EXAMPLE 5

| | |
|---|---|
| alpha amino n-butyrate | 1,000 mg |
| beta-hydroxy beta-methyl butyrate | 3,000 mg |
| DiCreatine Malate | 2,000 mg |

EXAMPLE 6

| | |
|---|---|
| alpha amino n-butyrate | 1,000 mg |
| beta-hydroxy beta-methyl butyrate | 3,000 mg |
| DiCreatine Malate | 2,000 mg |
| Arginine alpha-ketogluturate | 1,000 mg |
| L-Glutamine | 1,000 mg |
| Leucine alpha amino n-butyrate | 1,000 mg |

EXAMPLE 7

| | |
|---|---|
| alpha amino n-butyrate | 1,000 mg |
| alpha amino n-valerate | 1,000 mg |
| creatine alpha amino-n-butryate | 2,000 mg |
| Arginine alpha-ketogluturate | 1,000 mg |
| L-Glutamine | 1,000 mg |
| Leucine alpha amino n-butyrate | 1,000 mg |

EXAMPLE 8

| | |
|---|---|
| alpha amino n-valerate | 1,000 mg |
| beta-hydroxy beta-methyl butyrate | 3,000 mg |
| creatine alpha amino-n-butryate | 2,000 mg |

EXAMPLE 9

| | |
|---|---|
| alpha amino n-valerate | 1,000 mg |

A method of administering the disclosed dietary supplement to thereby increase lean body mass and/or preserve muscle mass may include orally administering to a mammal a therapeutically effective amount alpha amino n-butyrate. For example, the method may include the step of orally administering 0.01-100 mg/kg bodyweight of alpha amino n-butyrate, 0.01-100 mg/kg bodyweight of alpha amino n-valerate or 0.01-100 mg/kg bodyweight of a combination thereof.

In the alternative, the method of administering the disclosed dietary supplement to thereby increase lean body mass and/or preserve muscle mass may include the step of orally administering a therapeutically effective amount (e.g., 0.01-100 mg/kg bodyweight) of a salt, ether, ester or other derivative, with alpha amino n-butyrate, alpha amino-n-valerate, or a combination thereof. Further, the molar ratio of the branched chain amino acids salt, ether, ester or other derivative, and alpha amino n-butyrate or alpha amino-n-valerate may be 1:1 or 2:1

As a further alternative, the method of administering the disclosed dietary supplement to thereby increase lean body mass and/or preserve muscle mass may include the step of orally administering a therapeutically effective amount (e.g., 0.01-1000 mg/kg bodyweight) of a branched chain amino acid, and alpha amino n-butyrate or alpha amino-n-valerate or a combination thereof.

As a yet another alternative, the method of administering the disclosed dietary supplement to thereby increase lean body mass and/or preserve muscle mass may include the step of orally administering a therapeutically effective amount (e.g., 0.01-1000 mg/kg bodyweight) of a branched chain amino acid salt, ether, ester or other derivative and an alpha amino n-butyrate salt, ether, ester or other derivative. Further, the molar ratio of the branched chain amino acid salt, ether, ester or other derivative and alpha amino n-butyrate salt, ether, ester or other derivative may be 1:1 or 2:1.

In still another alternative, the method of administering the disclosed dietary supplement to thereby increase lean body mass and/or preserve muscle mass may include the step of orally administering a therapeutically effective amount (e.g., 0.01-1000 mg/kg bodyweight) of a branched chain amino acid salt, ether, ester or other derivative and an alpha amino-n-valerate salt, ether, ester or other derivative. Further, the molar ratio of the branched chain amino acid salt, ether, ester or other derivative and alpha amino-n-valerate salt, ether, ester or other derivative may be 1:1 or 2:1.

Having thus described certain embodiments of the invention, various other embodiments will become apparent to those having skill in the art that to no depart from the scope of the claims.

We claim:

1. A composition comprising alpha amino-n butyrate, alpha amino-n-valerate, or a combination of alpha amino-n butyrate and alpha amino-n-valerate.

2. The composition of claim 1, further comprising a branched chain amino acid.

3. The composition of claim 2, wherein the branched chain amino acid is selected from a group consisting of Leucine, Isoleucine and Valine.

4. The composition of claim 2, wherein the molar ratio of the branched chain amino acid and alpha amino n-butryate is selected from a group consisting of: 1:1 and 2:1.

5. The composition of claim 2, wherein the molar ratio of the branched chain amino acid and alpha amino n-valerate is selected from a group consisting of: 1:1 and 2:1.

6. The composition of claim 2, wherein the branched chain amino acid comprises a salt or other derivate.

7. The composition according to claim 1 wherein the composition is a salt or other derivate of alpha amino n-butyrate or alpha-amino-N-valerate.

8. The composition according to claim 1, wherein the composition is an ester or ether derivate of alpha amino n-butyrate or alpha-amino N-valerate.

9. A composition according to claim 2, wherein the composition is an ester or ether derivate of the branched chain amino acid.

10. The composition of claim 2, further comprising an agent selected from a group consisting of creatine, glutamine, threonine, methionine, tyrosine and alanine.

11. The method for increasing lean body mass and preserving muscle mass in a mammal comprising the steps of orally administering a therapeutically effective amount of a composition comprising alpha amino n-butryate or a salt, ether, ester or derivative thereof and alpha-amino n-valerate or a salt, ether, ester or derivative thereof.

12. The method of claim 10, wherein the composition further comprises a therapeutically effective amount of a branched chain amino acid or derivative thereof.

13. The method of claim 10, wherein the therapeutically effective amount of the alpha amino n-butryate or a salt, ether, ester or derivative thereof comprises 0.01-100 mg/kg bodyweight.

14. The method of claim 10, wherein the therapeutically effective amount of the alpha-amino n-valerate or a salt, ether, ester or derivative thereof comprises 0.01-100 mg/kg bodyweight.

15. The method of claim 10, wherein the therapeutically effective amount of the branched chain amino acid or derivative thereof comprises 0.01-100 mg/kg bodyweight.

16. A composition increasing lean body mass and preserving muscle mass including at least one of a consisting essentially of an alpha amino-n butyrate or an alpha amino-n-valerate.

17. The composition of claim 15 further consisting essentially of a branched chain amino acid.

18. The composition of claim 16, wherein the branched chain amino acid is selected from a group consisting of Leucine, Isoleucine and Valine.

19. The composition of claim 14, wherein the molar ratio of the branched chain amino acid and alpha amino n-butryate is selected from a group consisting of: 1:1 and 2:1.

20. The composition of claim 14, wherein the molar ratio of the branched chain amino acid and alpha amino n-valerate is selected from a group consisting of: 1:1 and 2:1.
